(19) United States
(12) Patent Application Publication
(10) Pub. No.: US 2026/0047805 A1
(43) Pub. Date: Feb. 19, 2026

(54) FLEXIBLY USABLE COUCH TOP FOR RT APPLICATIONS

(71) Applicant: Siemens Healthineers AG, Forchheim (DE)

(72) Inventors: Philipp AUDENRITH, Heiligenstadt (DE); Peter HASTREITER, Unterleinleiter (DE); Verena MUELLER-REINWALD, Gnotzheim (DE); Christoph DICKMANN, Nuernberg (DE)

(73) Assignee: Siemens Healthineers AG, Forchheim (DE)

(21) Appl. No.: 19/302,924

(22) Filed: Aug. 18, 2025

(30) Foreign Application Priority Data
Aug. 19, 2024 (DE) 20 2024 104 677.3

(51) Int. Cl. A61B 5/00; A61B 5/055; A61B 6/03; A61B 6/04

(52) U.S. Cl. CPC A61B 5/704; A61B 5/055; A61B 6/032; A61B 6/0407

(57) ABSTRACT

A couch top has an underside and an upper side. The underside and the upper side extend along a length in a longitudinal direction from an upper edge to a lower edge and across a width in a transverse direction from a left-hand edge to a right-hand edge. The couch top has left-hand and right-hand basic fixing elements. The left-hand basic fixing element is fastened to the couch top with a base member in the vicinity of the left-hand edge, and the right-hand basic fixing element is fastened with a base member in the vicinity of the right-hand edge. The base members of the basic fixing elements do not protrude beyond the underside, but the projections of the basic fixing elements do protrude beyond the underside. The basic fixing elements are fastened to the couch top such that their spacing in the transverse direction is variable.

### CROSS-REFERENCE TO RELATED APPLICATION(S)

The present application claims priority under 35 U.S.C. § 119 to German Patent Application No. 20 2024 104 677.3, filed Aug. 19, 2024, the entire contents of which are incorporated herein by reference.

### FIELD

One or more example embodiments of the present invention relate to a couch top,
wherein the couch top has an underside for laying the couch top on a patient table of a patient couch of a medical imaging modality and an upper side for laying a patient on the couch top,
wherein the underside and the upper side extend along a length in a longitudinal direction from an upper edge to a lower edge,
wherein the underside and the upper side extend across a width in a transverse direction from a left-hand edge to a right-hand edge.

### BACKGROUND

Independent of the grammatical term usage, individuals with male, female or other gender identities are included within the term.

Such a couch top is known for example from U.S. Pat. No. 7,076,821 B2.

A patient table of a patient couch of a medical imaging modality (for example an MR system or a CT system) conventionally has a soft, trough-like curved surface. The trough-like embodiment makes it possible to position a patient reliably on the patient table. The soft surface allows the patient to lie comfortably.

This poses no problem for purely diagnostic purposes. Medical imaging modalities are, however, also used for planning radiotherapy. When it comes to precisely planning radiotherapy, it is important for the patient to be positioned right from the planning stage in precisely the same place on the patient table as that in which the patient will subsequently be positioned when the radiotherapy is carried out. For radiotherapy planning purposes, a flat, hard couch top (also often denoted overlay) is laid on the patient table and the patient is then positioned on the overlay. In order to precisely position the couch top on the patient table, fixing elements that interact with corresponding counter-elements on the patient table are arranged on the underside of the couch top.

In many patient tables, the counter-elements are configured as groove or channel-like recesses that extend in the longitudinal direction of the patient table. The spacing of the recesses from one another may, however, vary from patient table to patient table. A couch top designed for a specific patient table can therefore typically only be laid on this patient table (or a similar patient table) but not on other types of patient table.

### SUMMARY

An object of one or more example embodiments of the present invention is accordingly to further develop a couch top in such a manner that it can be used on various patient tables.

At least this object is achieved by a couch top having the features of the independent claim(s). Advantageous embodiments of the couch top according to one or more example embodiments of the present invention are the subject matter of the dependent claims.

According to one or more example embodiments of the present invention, a couch top of the above-stated type is embodied in that
the couch top has a left-hand and a right-hand basic fixing element,
the basic fixing elements have a respective base member and a respective projection,
the left-hand basic fixing element is fastened to the couch top with its base member in the vicinity of the left-hand edge and the right-hand basic fixing element is fastened to the couch top with its base member in the vicinity of the right-hand edge, such that the base members of the basic fixing elements do not protrude beyond the underside and the projections of the basic fixing elements do protrude beyond the underside, and
the basic fixing elements are fastened to the couch top in such a manner that their spacing from one another in the transverse direction is variable.

Since the couch top is used for positioning an adult person, the length corresponds to about the body size of an adult person and the width to about the width of an adult person.

The projections of the basic fixing elements can be introduced into the groove or channel-like recesses that are often present in the patient table and extend in the longitudinal direction of the patient table. Thanks to the possibility of being able to vary the spacing of the basic fixing elements from one another in the transverse direction, the spacing of the basic fixing elements from one another in the transverse direction can be adapted to the spacing of the groove or channel-like recesses in the patient table. As a result, the couch top can (within certain limits) be used irrespective of the spacing of the groove or channel-like recesses from one another.

It is possible for both basic fixing elements to be adjustable in the transverse direction. Preferably, however, one of the two basic fixing elements is fixed in the transverse direction relative to the couch top and just the other of the two basic fixing elements is adjustable in the transverse direction. This embodiment on the one hand simplifies handling, as it is clear from the outset which basic fixing element has to be repositioned in order to adjust the spacing in the transverse direction. On the other hand, clearer positioning of the couch top on the patient table in the transverse direction is obtained.

It is possible for this other of the two basic fixing elements to be fastened to the couch top in such a manner that it is adjustable in the transverse direction without its position in the longitudinal direction being modified. Preferably, however, this other of the two basic fixing elements is fastened to the couch top in such a manner that adjustment of this basic fixing element in the transverse direction is necessarily accompanied by adjustment of this basic fixing element in the longitudinal direction. This embodiment offers both structural and handling advantages.

In order to achieve such adjustability, the couch top may have a limit stop edge that, viewed in a plane defined by the longitudinal direction and the transverse direction, extends at an angle other than 0° and 90°, i.e., obliquely, to the longitudinal direction. In this case, in the fastened state on the limit stop edge, the other of the two basic fixing elements is displaceable along the limit stop edge for adjustment. The angle relative to the longitudinal direction is typically an angle of between 30° and 60°, in particular between 40° and 50°, for example approx. 45°.

On fixation of this other of the two basic fixing elements to the limit stop edge, threaded bores for fastening screws can be introduced at discrete locations on the limit stop edge and the base member of the other of the two basic fixing elements may have an elongate hole that extends in the direction of the limit stop edge. As a result, although the position of the fastening screws along the limit stop edge is fixed, once the fastening screws have been loosened, the corresponding basic fixing element can be displaced along the limit stop edge and fixed in place again in the new position by retightening the fastening screws.

As already mentioned, it is possible for the one of the two basic fixing elements likewise to be adjustable in the transverse direction. It is likewise also possible for the one of the two basic fixing elements to be completely non-adjustable, i.e., neither in the transverse direction nor in the longitudinal direction. Preferably, however, the one of the two basic fixing elements is adjustable in the longitudinal direction without its spacing from the other of the two basic fixing elements in the transverse direction being modified. For example, a (further) limit stop edge of the couch top, to which edge the one of the two basic fixing elements is fixed, can extend in the longitudinal direction.

It is possible for the position of the one of the two basic fixing elements to be continuously variable in the longitudinal direction. However, the one of the two basic fixing elements is preferably fixable to the couch top solely at discrete positions viewed in the longitudinal direction. For example, the further limit stop edge may have threaded bores for fastening screws, wherein the number of threaded bores is greater than the number of fastening screws. Depending on which of the threaded bores have the fastening screws inserted and which remain empty, the one of the two basic fixing elements can be fixed in one or the other (and possibly also further) positions.

The one of the two basic fixing elements preferably has on its projection a lug that projects in the transverse direction. This embodiment is in particular advantageous with the option for the one of the two basic fixing elements to be fixed solely at discrete positions viewed in the longitudinal direction to the couch top.

The spacing of the basic fixing elements from one another in the transverse direction is preferably continuously variable. This is in particular advantageous because it makes it straightforwardly possible to compensate for any manufacturing tolerances that might result in slight variations in the spacing of the groove or channel-like recesses from patient table to patient table of the same type. Continuous adjustability may be achieved, for example, in conjunction with the obliquely extending limit stop edge already described above.

In some cases, solely the basic fixing elements are present. However, it is preferably provided that,
in addition to the basic fixing elements, the couch top has an additional left-hand and an additional right-hand fixing element that, viewed in the longitudinal direction, are spaced from the basic fixing elements,
the additional fixing elements have a respective base member and a respective projection,
the additional left-hand fixing element is fastened to the couch top with its base member in the vicinity of the left-hand edge and the additional right-hand fixing element is fastened to the couch top with its base member in the vicinity of the right-hand edge, such that the base members of the additional fixing elements do not protrude beyond the underside and the projections of the additional fixing elements do protrude beyond the underside, and
the additional fixing elements are fastened to the couch top in such a manner that their spacing from one another in the transverse direction is variable.

As a result, positioning and fixation of the couch top on the patient table can be distinctly improved.

In contrast with the basic fixing elements, both additional fixing elements are preferably adjustable relative to the couch top viewed in the transverse direction. As a result, the orientation of the couch top on the patient table can be varied or corrected as required. Both additional fixing elements are furthermore preferably adjustable in the transverse direction without their position in the longitudinal direction being modified. This offers structural advantages.

The two basic fixing elements are typically arranged roughly in the same (in substantially the same) basic position viewed in the longitudinal direction. This is the case irrespective of whether the additional fixing elements are or are not present. The basic position is usually determined in such a manner that, in the case of an adult person, the shoulders lie roughly in the basic position.

If the two additional fixing elements are present, they are typically arranged roughly in the same (in substantially the same) additional position viewed in the longitudinal direction. If the basic position is determined in such a manner that, in the case of an adult person, the shoulders lie roughly at the basic position, the additional position is often determined such that, in the case of an adult person, the feet lie roughly in the additional position.

### BRIEF DESCRIPTION OF THE DRAWINGS

The above-described characteristics, features and advantages of this invention and the manner in which these are achieved will become clearer and more distinctly comprehensible from the following description of the exemplary embodiments which are explained in greater detail in connection with the drawings, in which in schematic representation:

FIG. 1 shows a medical imaging modality,

FIG. 2 is a plan view from above onto a couch top,

FIG. 3 is a perspective view of an underside of a couch top,

FIG. 4 shows a detail of FIG. 3,

FIG. 5 is a plan view onto a basic fixing element,

FIG. 6 shows a further detail of FIG. 3

FIG. 7 is a view of the couch top viewed from the foot end.

### DETAILED DESCRIPTION

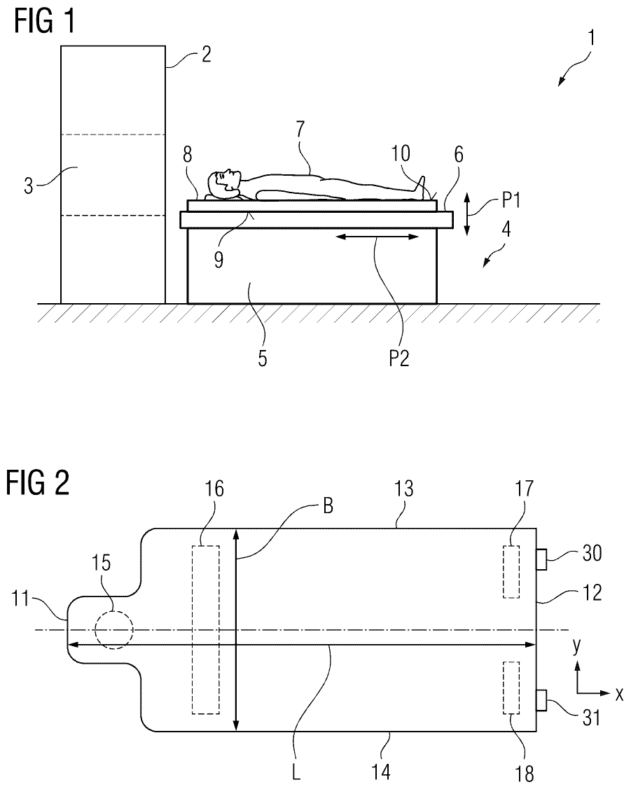

According to FIG. 1, a medical imaging modality 1 (for example a CT system or an MR system) has an annular base member 2 that defines a substantially cylindrical examination volume 3. A patient couch 4 is arranged in front of the base member 2. The patient couch 4 has a substructure 5 and a patient table 6. The patient table 6 is arranged height-adjustably on the substructure 5. This is indicated in FIG. 1 by a double-headed arrow P1. The patient table 6 is also arranged longitudinally displaceably on the substructure 5. This is indicated in FIG. 1 by a double-headed arrow P2.

In order to investigate a patient 7, a couch top 8 is laid with its underside 9 on the patient table 6. In this state, the patient 7 is laid on an upper side 10 of the couch top 8. The patient 7 is typically an adult person.

The couch top 8 is thus intended for the patient 7 to lie on it. The dimensions of the couch top 8 are determined accordingly. Specifically, the underside 9 and the upper side 10 extend according to FIG. 2 in a longitudinal direction x from an upper edge 11 to a lower edge 12 along a length L. The length L is dimensioned in such a manner that the patient 7 (viewed in the longitudinal direction x) is located entirely in the region of the couch top 8. The length L is thus of the order of approx. 200 cm. The underside 9 and the upper side 10 likewise extend in a transverse direction y from a left-hand edge 13 to a right-hand edge 14 across a width B. The width B is dimensioned in such a manner that the patient 7 (viewed in the transverse direction y) is located entirely in the region of the couch top 8. The width B is thus within the range between 50 cm and 70 cm, for example approx. 60 cm. The edges 13, 14 are denoted left-hand and right-hand edges 13, 14 respectively because the patient 7 lies with their back on the couch top 8. FIG. 2 furthermore schematically indicates regions 15 to 18. The head of the patient 7 is arranged roughly in region 15, the shoulders of the patient 7 roughly in region 16 and the two feet of the patient 7 roughly in regions 17 and 18.

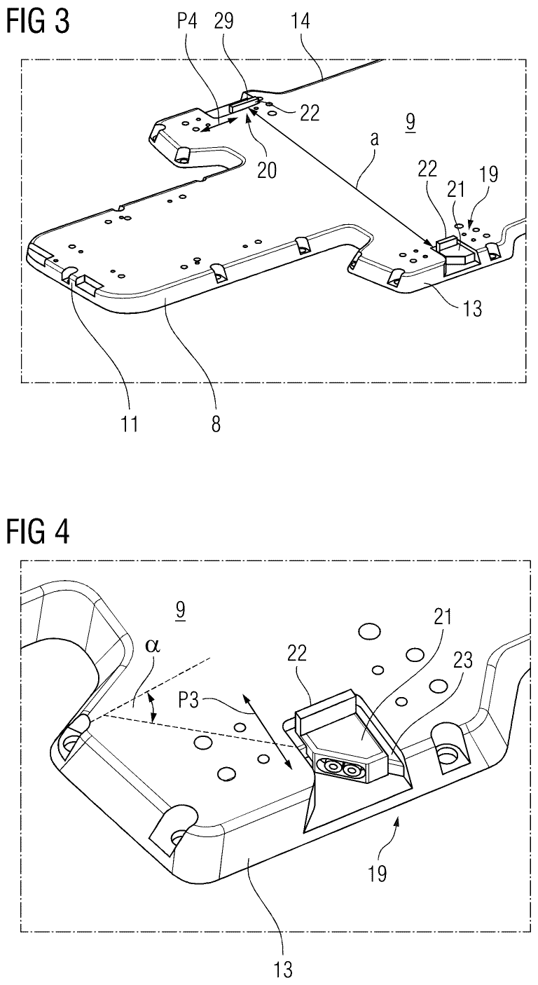

According to FIG. 3, the couch top 8 has a left-hand basic fixing element 19 and a right-hand basic fixing element 20. In the portrayal of FIG. 3, the left-hand basic fixing element 19 is arranged on the right and the right-hand basic fixing element 20 on the left. This is because the couch top 8 is portrayed as it were turned over, i.e., the underside 9 is on top in the portrayal of FIG. 3. As is apparent, the two basic fixing elements 19, 20 are arranged roughly in the same basic position viewed in the longitudinal direction x. The basic position is determined in such a manner that, in the case of an adult person, the shoulders lie roughly in the basic position when the person is lying on the couch top.

The two basic fixing elements 19, 20 have a respective base member 21 and a respective projection 22. The left-hand basic fixing element 19 is fastened to the couch top 8 with its base member 21 in the vicinity of the left-hand edge 13. Similarly, the right-hand basic fixing element 20 is fastened to the couch top 8 with its base member 21 in the vicinity of the right-hand edge 14. Fastening is such that the base members 21 of the basic fixing elements 19, 20 do not protrude beyond the underside 9 and the projections 22 of the basic fixing elements 19, 20 do protrude beyond the underside 9. For example, the couch top 8 can have on its underside 9 corresponding recesses into which the base members 21 can descend.

The basic fixing elements 19, 20 are fastened to the couch top 8 in such a manner that their spacing a from one another in the transverse direction y is variable. Specifically, the right-hand basic fixing element 20 is fixed relative to the couch top 8 in the transverse direction y. The left-hand basic fixing element 19, on the other hand, is adjustable in the transverse direction y. This is indicated by a double-headed arrow P3 in FIG. 4, which is a portrayal of the couch top 8 in the region of the left-hand basic fixing element 19.

Specifically, the left-hand basic fixing element 19 is fastened to the couch top 8 in such a manner that adjustment of the left-hand basic fixing element 19 in the transverse direction y is necessarily accompanied by adjustment of the left-hand basic fixing element 19 in the longitudinal direction x. In particular, according to the portrayal in FIGS. 3 and 4, the couch top 8 has a limit stop edge 23 that, viewed in a plane defined by the longitudinal direction x and the transverse direction y, extends at an angle α other than 0° and 90° to the longitudinal direction x. The angle α is preferably between 30° and 60°, in particular of the order of approx. 45°.

In the fastened state, the left-hand basic fixing element 19 rests against the limit stop edge 23. It can be displaced along the limit stop edge 23 for adjustment. For example, the base member 21 of the left-hand basic fixing element 19 may have an elongate hole 24 corresponding to the portrayal in FIG. 5 through which two fastening screws 25 are screwed into corresponding threaded bores that are arranged in the limit stop edge 23. If the fastening screws 25 are loosened, the left-hand basic fixing element 19 can be displaced along the limit stop edge 23. If the fastening screws 25 retightened, the left-hand basic fixing element 19 is fixed in place. Thanks to this kind of fastening and adjustability, the spacing a of the basic fixing elements 19, 20 from one another in the transverse direction y is in particular also continuously variable.

As already mentioned, the right-hand basic fixing element 20 is not adjustable in the transverse direction y. However, as indicated by a double-headed arrow P4 in FIG. 3, it is adjustable in the longitudinal direction x. The spacing a in the transverse direction y from the left-hand basic fixing element 19 is not modified as a result.

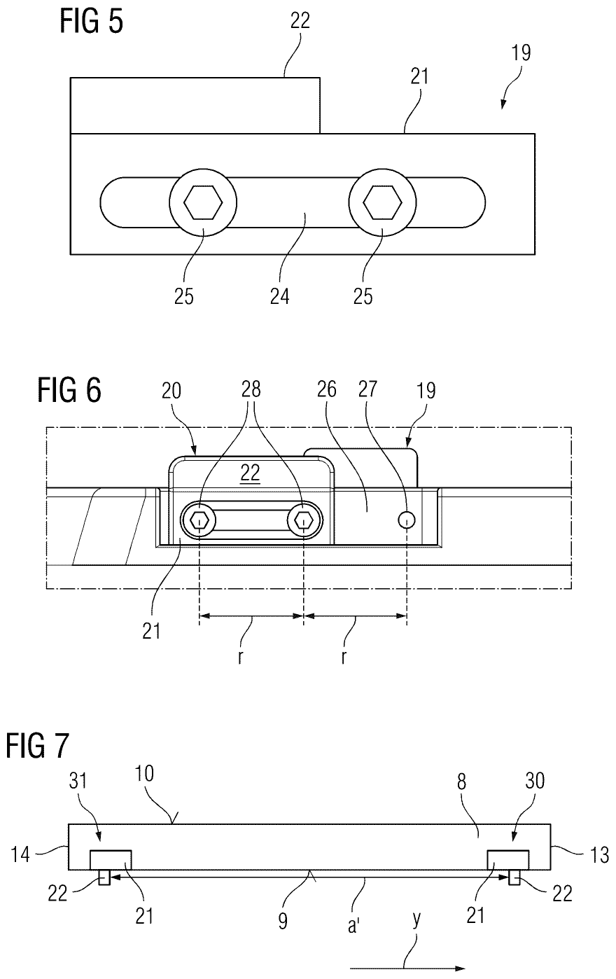

The right-hand basic fixing element 20 is fixable to the couch top 8 solely at discrete positions viewed in the longitudinal direction x. For example, according to the FIGS. 3 and 6, the couch top 8 may have a further limit stop edge 26 that extends parallel to the longitudinal direction x in the vicinity of the right-hand edge 14. Threaded bores 27 can, for example, be introduced into the further limit stop edge 26, which bores are spaced apart from one another in the longitudinal direction x in a uniform pattern r. If, for example and as shown in FIG. 6, three threaded bores 27 are present, a respective fixing screw 28 can be screwed into the middle and one of the two outer threaded bores 27. As a result, the two fastening screws 28 can fix the right-hand basic fixing element 20 in either a front or a rear position viewed in the longitudinal direction x.

According to FIG. 3, the right-hand basic fixing element 20 has on its projection 22 a lug 29 that projects in the transverse direction y. Precise positioning of the couch top 8 on the patient table 6 in the longitudinal direction x can be achieved by way of the lug 29. This applies in particular if the right-hand basic fixing element 20 can only be fixed to the couch top 8 in the defined positions in the longitudinal direction x.

In addition to the basic fixing elements 19, 20, the couch top 8 preferably has a left-hand fixing element 30 and a right-hand fixing element 31. Viewed in the longitudinal direction x, the two additional fixing elements 30, 31 are spaced from the basic fixing elements 19, 20. The two additional fixing elements 30, 31 are typically arranged roughly in the same additional position viewed in the longitudinal direction x. The additional position is usually determined in such a manner that, in the case of an adult person, the feet lie roughly in the additional position when the person is lying on the couch top 8.

According to FIG. 7, the additional fixing elements 30, 31 likewise have, similarly to the basic fixing elements 19, 20, a respective base member 21 and a respective projection 22. The arrangement is also completely similar. In particular, the additional left-hand fixing element 30 is fastened to the couch top 8 with its base member 21 in the vicinity of the left-hand edge 13 and the additional right-hand fixing element 31 is fastened to the couch top 8 with its base member 21 in the vicinity of the right-hand edge 14. Fastening is also such that the base members 21 of the additional fixing elements 30, 31 do not protrude beyond the underside 9 and the projections 22 of the additional fixing elements 30, 31 do protrude beyond the underside 9. Finally, fastening of the additional fixing elements 30, 31 to the couch top 8 is also such that their spacing a′ from one another is variable in the transverse direction y. However, in contrast with the basic fixing elements 19, 20, both additional fixing elements 30, 31 are adjustable relative to the couch top 8 viewed in the transverse direction y. On adjustment in the transverse direction y, the two additional fixing elements 30, 31 preferably do not, however, change their position in the longitudinal direction x. Adjustability may, for example, be similar to the adjustment of the left-hand basic fixing element 19. It is merely necessary to orient the corresponding limit stop edges in such a manner that they extend parallel to the transverse direction y.

One or more example embodiments of the present invention have many advantages. In particular, the couch top 8 is virtually universally applicable to and usable for different patient tables 6.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of the embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing embodiments. The present invention may, however, be embodied in many alternate forms and should not be construed as limited to only the embodiments set forth herein.

Although the present invention has been illustrated and described in greater detail with reference to the preferred exemplary embodiments, the present invention is not restricted by the disclosed examples and other variations may be derived therefrom by a person skilled in the art without going beyond the scope of protection of the present invention.

What is claimed is:

1. A couch top, comprising:
an underside for laying the couch top on a patient table of a patient couch of a medical imaging modality; and
an upper side for laying a patient on the couch top; wherein
the underside and the upper side extend along a length in a longitudinal direction from an upper edge to a lower edge,
the underside and the upper side extend across a width in a transverse direction from a left-hand edge to a right-hand edge,
the couch top has a left-hand basic fixing element and a right-hand basic fixing element,
each of the left-hand basic fixing element and the right-hand basic fixing element have a respective base member and a respective projection,
the left-hand basic fixing element is fastened to the couch top with the respective base member in a vicinity of the left-hand edge,
the right-hand basic fixing element is fastened to the couch top with the respective base member in a vicinity of the right-hand edge,
the respective base member of the left-hand basic fixing element and the respective base member of the right-hand basic fixing element do not protrude beyond the underside, whereas the respective projection of the left-hand basic fixing element and the respective projection of the right-hand basic fixing element do protrude beyond the underside, and
the left-hand basic fixing element and the right-hand basic fixing element are fastened to the couch top such that a spacing between the left-hand basic fixing element and the right-hand basic fixing element is variable in the transverse direction.

2. The couch top as claimed in claim 1, wherein
one of the left-hand basic fixing element or the right-hand basic fixing element is fixed in the transverse direction relative to the couch top, and
another of the left-hand basic fixing element or the right-hand basic fixing element is adjustable in the transverse direction.

3. The couch top as claimed in claim 2, wherein the other of the left-hand basic fixing element or the right-hand basic fixing element is fastened to the couch top such that adjustment of the other of the left-hand basic fixing element or the right-hand basic fixing element in the transverse direction is necessarily accompanied by adjustment of the other of the left-hand basic fixing element or the right-hand basic fixing element in the longitudinal direction.

4. The couch top as claimed in claim 3, wherein
the couch top has a limit stop edge that, viewed in a plane defined by the longitudinal direction and the transverse direction, extends at an angle other than 0° or 90° relative to the longitudinal direction, and
the other of the left-hand basic fixing element or the right-hand basic fixing element, in a fastened state, rests against the limit stop edge and is displaceable along the limit stop edge for adjustment.

5. The couch top as claimed in claim 2, wherein the one of the left-hand basic fixing element or the right-hand basic fixing element is adjustable in the longitudinal direction without a spacing from the other of the left-hand basic fixing element or the right-hand basic fixing element in the transverse direction being modified.

6. The couch top as claimed in claim 5, wherein the one of the left-hand basic fixing element or the right-hand basic fixing element is fixable to the couch top only at discrete positions viewed in the longitudinal direction.

7. The couch top as claimed in claim 2, wherein
the one of the left-hand basic fixing element or the right-hand basic fixing element has a lug on the respective projection, and
the lug projects in the transverse direction.

8. The couch top as claimed in claim 1, wherein the spacing between the left-hand basic fixing element and the right-hand basic fixing element in the transverse direction is continuously variable.

9. The couch top as claimed in claim 1, further comprising:
an additional left-hand fixing element and an additional right-hand fixing element that, viewed in the longitudinal direction, are spaced apart from the left-hand basic fixing element and the right-hand basic fixing element, wherein
each of the additional left-hand fixing element and the additional right-hand fixing element have a respective base member and a respective projection,
the additional left-hand fixing element is fastened to the couch top with the respective base member in the vicinity of the left-hand edge and the additional right-hand fixing element is fastened to the couch top with the respective base member in the vicinity of the right-hand edge,
the respective base member of each of the additional left-hand fixing element and the additional right-hand fixing element does not protrude beyond the underside, whereas the respective projection of each of the additional left-hand fixing element and the additional right-hand fixing element does protrude beyond the underside, and
the additional left-hand fixing element and the additional right-hand fixing element are fastened to the couch top such that a spacing between the additional left-hand fixing element and the additional right-hand fixing element is variable in the transverse direction.

10. The couch top as claimed in claim 9, wherein the additional left-hand fixing element and the additional right-hand fixing element are adjustable relative to the couch top viewed in the transverse direction.

11. The couch top as claimed in claim 10, wherein both the additional left-hand fixing element and the additional right-hand fixing element are adjustable in the transverse direction without a position in the longitudinal direction being modified.

12. The couch top as claimed in claim 9, wherein
the left-hand basic fixing element and the right-hand basic fixing element are arranged in substantially the same basic position viewed in the longitudinal direction, and
the additional left-hand fixing element and the additional right-hand fixing element are arranged in substantially the same additional position viewed in the longitudinal direction.

13. The couch top as claimed in claim 12, wherein, in case of an adult person, shoulders of the adult person lie in a basic position and feet of the adult person lie in an additional position when the adult person is lying on the couch top.

14. The couch top as claimed in claim 1, wherein the left-hand basic fixing element and the right-hand basic fixing element are arranged in substantially the same basic position viewed in the longitudinal direction.

15. The couch top as claimed in claim 14, wherein, in case of an adult person, shoulders of the adult person lie in the basic position when the adult person is lying on the couch top.

16. The couch top as claimed in claim 3, wherein the one of the left-hand basic fixing element or the right-hand basic fixing element is adjustable in the longitudinal direction without a spacing from the other of the left-hand basic fixing element or the right-hand basic fixing element in the transverse direction being modified.

17. The couch top as claimed in claim 4, wherein the one of the left-hand basic fixing element or the right-hand basic fixing element is adjustable in the longitudinal direction without a spacing from the other of the left-hand basic fixing element or the right-hand basic fixing element in the transverse direction being modified.

18. The couch top as claimed in claim 3, wherein
the one of the left-hand basic fixing element or the right-hand basic fixing element has a lug on the respective projection, and
the lug projects in the transverse direction.

19. The couch top as claimed in claim 2, wherein the spacing between the left-hand basic fixing element and the right-hand basic fixing element in the transverse direction is continuously variable.

20. The couch top as claimed in claim 3, wherein the spacing between the left-hand basic fixing element and the right-hand basic fixing element in the transverse direction is continuously variable.
